(19) United States
(12) Patent Application Publication
(10) Pub. No.: US 2013/0274534 A1
(43) Pub. Date: Oct. 17, 2013

(54) PARAFFIN DISPROPORTIONATION WITH ZEOLITE Y

(71) Applicant: Christopher S. Gill, Katy, TX (US)

(72) Inventor: Christopher S. Gill, Katy, TX (US)

(73) Assignee: PHILLIPS 66 COMPANY, Houston, TX (US)

(21) Appl. No.: 13/787,925

(22) Filed: Mar. 7, 2013

Related U.S. Application Data
(60) Provisional application No. 61/623,808, filed on Apr. 13, 2012.

(51) Int. Cl. B01J 29/08; C07C 6/10

(52) U.S. Cl. CPC B01J 29/088; B01J 29/084; C07C 6/10
USPC 585/708

(57) ABSTRACT

Methods relate to disproportionation of hydrocarbons utilizing a zeolite catalyst. The methods include reacting pentanes in contact with ultrastable zeolite Y having a silica to alumina ratio of less than 80 to disproportionate the pentanes into butanes and hexanes. The ultrastable zeolite Y is defined by having a sodium oxide content of less than 1% by weight.

### CROSS-REFERENCE TO RELATED APPLICATIONS

This application is a non-provisional application which claims benefit under 35 USC §119(e) to U.S. Provisional Application Ser. No. 61/623,808 filed Apr. 13, 2012, entitled “Paraffin Disproportionation with Zeolite Y,” which is incorporated herein in its entirety.

### STATEMENT REGARDING FEDERALLY SPONSORED RESEARCH OR DEVELOPMENT

None.

### FIELD OF THE INVENTION

Embodiments of the invention relate to disproportionation of hydrocarbons utilizing a zeolite catalyst.

### BACKGROUND OF THE INVENTION

Future crude slate and regulatory changes may displace high Reid vapor pressure (RVP) gasoline blend components from the gasoline pool. Disproportionation reactions allow conversion of high RVP pentanes into heavier gasoline and alkylate feed. Converted products can thus be returned to the gasoline pool as desired.

Several catalysts exist for the disproportionation. For example, some zeolite compositions, aluminum chloride based compounds and heteropolyacids provide catalytic activity. However, such prior catalysts that can be expensive also often tend to cause undesired cracking and lack desired lifetimes, selectivity or activity. Further, these catalysts may add to costs by requiring dry conditions with water levels of parts per million or below or by necessitating complex equipment when the catalyst is a liquid acid and not a solid phase composition.

Therefore, a need exists for catalyst utilized for disproportionation of paraffins.

### BRIEF SUMMARY OF THE DISCLOSURE

In one embodiment, a method of disproportionating hydrocarbons includes reacting pentanes in contact with ultrastable zeolite Y having a silica to alumina ratio of less than 80. The reacting converts the pentanes into butanes and hexanes. The ultrastable zeolite Y may be defined by having a sodium oxide (Na₂O) content of less than 1% by weight.

### DETAILED DESCRIPTION

Turning now to the detailed description of the preferred arrangement or arrangements of the present invention, it should be understood that the inventive features and concepts may be manifested in other arrangements and that the scope of the invention is not limited to the embodiments described or illustrated. The scope of the invention is intended only to be limited by the scope of the claims that follow.

Methods relate to disproportionation of hydrocarbons utilizing a zeolite catalyst. For some embodiments, the hydrocarbons include paraffins having from 3 to 5 carbons, such as pentane. The methods include reacting the pentanes in contact with ultrastable zeolite Y to disproportionate the pentanes into butanes and hexanes.

In some embodiments, a sodium oxide content of less than 1% by weight defines the ultrastable zeolite Y, which has a silica to alumina ratio of less than 80, less than 10 or at 5.2. In order to enhance the thermal stability of zeolite Y and make the ultrastable zeolite Y, manufacturers may steam the zeolite Y at temperatures above 525° C., causing aluminum oxygen bond cleavage and subsequent dealumination of the zeolite framework. Such a process does not change the bulk silica/alumina ratio, but does create extra-framework aluminum species since the aluminum is expelled from the framework. The ultrastable zeolite Y contains hydrogen counter ions for some embodiments and is thus in proton form. Other embodiments utilize the ultrastable zeolite Y that is rare earth element exchanged, such as lanthanum exchanged.

In operation, the pentanes enter a reactor containing the catalyst between 200° C. and 400° C. and 1,500 kilopascals (kPa) and 4,250 kPa. The temperature and pressure in some embodiments is at 250° C. and at 2859 kilopascals. Separation of resulting products into the butanes and the hexanes allows for use of the butanes as feed into an alkylation unit and return of the hexanes to the gasoline pool. Any remaining pentanes can recycle back through the reactor or also be blended in the gasoline pool if sufficient RVP decrease is achieved.

For some embodiments, the reaction has a selectivity that is at least 89%, as defined by mass of the butanes and the hexanes divided by total non-pentane product mass. The reacting that takes place in one pass through the reactor converts at least 20% of the pentanes in some embodiments. A mol ratio of the butanes (C4) to the hexanes (C6) produced by the reacting may be between 0.7 and 1.2 or less than 1.0 with over 90% of the butanes produced being isobutane.

The C4/C6 mol ratio and isobutane purity within the butanes provide an indication of wanted operating conditions and product properties. The C4/C6 mol ratio if above one provides an indication of undesired cracking reactions that convert higher weight components (≧C6) into lighter weight components (≦C4). Therefore, level of the cracking reactions thus corresponds to how much the C4/C6 mol ratio exceeds one. The C4/C6 mol ratio if below one indicates alkylation reactions are building larger molecules from the butanes or the butanes are being consumed to form unwanted surface coke, which diminishes catalyst lifetime. The C4/C6 mol ratio may thus approach zero with increasing coking level. Increases in concentration of the isobutanes in the butanes toward or above market isobutane purity (>95%) facilitates using the butanes for alkylate feed.

Some embodiments further include regenerating the ultrastable zeolite Y by burning off the surface coke deposited during the reacting of the pentanes. Such regeneration may occur in a continuous or semi-continuous approach. Exemplary temperatures suitable for regeneration range from 400° C. to 600° C.

The following examples of certain embodiments of the invention are given. Each example is provided by way of explanation of the invention, one of many embodiments of the invention, and the following examples should not be read to limit, or define, the scope of the invention.

Catalytic tests were performed in a packed bed, down-flow tube reactor. Each test used about 18 mL of respective catalyst mixed with about 7 mL alundum, which mixture was packed in the reactor between layers of glass wool and glass beads (3 mm). Feed of 50/50 wt % pentane/isopentane was supplied to the reactor at 16 milliliters per hour (mL/hr). Weight hourly space velocity was 0.9 hr⁻¹. The reactor temperature and pressure was regulated to about 2859 Pa and about 250° C.

Reactor products were quantified using a gas chromatograph (GC) equipped with a flame ionization detector (FID). Catalyst performance was calculated based on the GC data in terms of conversion, selectivity to butane and hexane products, C4/C6 mol ratio and isobutane purity of butane products. Results are shown after four hours on stream in a following table for each of six catalyst tests including ultrastable zeolite Y in proton form (H-USY) with a silica to alumina ratio of either 5.2 or 30.0, lanthanum exchanged ultrastable zeolite Y (La-USY), comparative non-ultrastable zeolite Y (H-Y), comparative potassium exchanged ultrastable zeolite Y (K-USY) and comparative ZSM-5 in proton form (H-ZSM-5).

| | Na2O | SiO2/ | | | | |
|---|---|---|---|---|---|---|
| Zeolite | Wt. | Al2O3 | Conversion | Selectivity | C4/C6 | % IC4 |
| Catalyst | % | Ratio | (%) | (%) | Ratio | of C4 |
| H-USY | <1 | 5.2 | 20 | 89 | 0.7 | 93 |
| La-USY | <1 | 5.2 | 20 | 90 | 0.9 | 93 |
| H-USY | <1 | 30 | 10 | 90 | 0.4 | 94 |
| H-Y | >1 | 5.1 | 0 | — | — | — |
| K-USY | <1 | 5.2 | 0 | — | — | — |
| H-ZSM-5 | <1 | 50 | 28 | 62 | 1.6 | 52 |

Since ultrastable zeolite Y was purchased in an ammonium form, the zeolite required calcination to convert to the H-USY. Drying and calcination steps were performed under one liter per minute of flowing air and at 150° C. for 2 hrs and then raised 5° C. per minute to 450° C. for 14 hrs. In the test, the H-USY generated a C4/C6 mol ratio of 0.4, which is indicative of production of heavier and less volatile gasoline blend components from pentanes in order to achieve desired RVP reduction. Furthermore, the H-USY generated higher isobutane purity than the H-ZSM-5.

The H-USY and La-USY generated butane products far from the thermodynamic equilibrium of 51% isobutane at 250° C. However, the H-ZSM-5 generated products close to the thermodynamic equilibrium. High isobutane purity indicated the catalysts were producing the kinetic products for the reaction and were not active for the isomerization reaction between n-butane and isobutane.

The La-USY was also synthesized from ammonium ultrastable zeolite Y stirred in a solution of lanthanum nitrate (0.2 molar (M), 2 hrs, 80° C., 11 milliliters per gram) and then filtered (500 mL deionized (DI) water). One third of the mass was removed and saved. The exchange procedure was repeated for a total of three times, each time saving one third of the initial mass. The samples were dried overnight (80° C., 1 L/min flowing air) and calcined as described with the H-USY prior to testing.

Coked H-USY was also regenerated via calcination (150° C. for 2 hr, 5° C./min ramp rate, X° C. for 14 hr, 1 L/min flowing air, where X=400° C., 500° C. and 600° C.). These regenerated catalysts were then tested in the reactor under same conditions as the fresh catalyst tests to determine recyclability. The isobutane purity was constant at about 92% for all regenerated catalysts. The catalysts calcined at 400° C. and 500° C. displayed similar activity profiles compared to fresh catalyst, while the 600° C. sample displayed lower activities at all times. As the regeneration temperature increased, the resulting C4/C6 mol ratios increased from 0.93 up to 1.70, indicating the cracking was enhanced by higher regeneration temperatures. Only a partial regeneration of the catalyst may be desired for some embodiments, since the 400° C. regenerated catalyst displayed identical conversion but lower C4/C6 mol ratios than the fresh catalyst.

In closing, it should be noted that the discussion of any reference is not an admission that it is prior art to the present invention, especially any reference that may have a publication date after the priority date of this application. At the same time, each and every claim below is hereby incorporated into this detailed description or specification as an additional embodiment of the present invention.

Although the systems and processes described herein have been described in detail, it should be understood that various changes, substitutions, and alterations can be made without departing from the spirit and scope of the invention as defined by the following claims. Those skilled in the art may be able to study the preferred embodiments and identify other ways to practice the invention that are not exactly as described herein. It is the intent of the inventors that variations and equivalents of the invention are within the scope of the claims while the description, abstract and drawings are not to be used to limit the scope of the invention. The invention is specifically intended to be as broad as the claims below and their equivalents.

1. A method, comprising:
reacting pentanes in contact with ultrastable zeolite Y having a silica to alumina ratio of less than 80 to disproportionate the pentanes into butanes and hexanes, wherein the ultrastable zeolite Y is defined by having a sodium oxide content of less than 1% by weight.

2. The method of claim 1, wherein the ultrastable zeolite Y is in proton form containing hydrogen counter ions.

3. The method of claim 1, wherein the ultrastable zeolite Y has a silica to alumina ratio of less than 10.

4. The method of claim 1, wherein the ultrastable zeolite Y has a silica to alumina ratio of 5.2.

5. The method of claim 1, wherein the ultrastable zeolite Y is rare earth element exchanged.

6. The method of claim 1, wherein the ultrastable zeolite Y is lanthanum exchanged.

7. The method of claim 1, wherein the reacting is at a temperature between 100° C. and 400° C. and at a pressure between 1,500 kilopascals and 4,250 kilopascals.

8. The method of claim 1, wherein the reacting is at 250° C. and at 2859 kilopascals.

9. The method of claim 1, wherein the reacting has a selectivity that is defined as mass of the butanes and the hexanes divided by total non-pentane product mass and is at least 89%.

10. The method of claim 1, wherein the reacting converts at least 20% of the pentanes.

11. The method of claim 1, wherein a mol ratio of the butanes to the hexanes produced by the reacting is less than 1.

12. The method of claim 1, wherein over 90% of the butanes produced by the reacting are isobutane.

13. The method of claim 1, wherein the reacting converts at least 20% of the pentanes, a mol ratio of the butanes to the hexanes produced by the reacting is less than 1 and over 90% of the butanes produced by the reacting are isobutanes.

14. The method of claim 1, further comprising regenerating the ultrastable zeolite Y by burning off coke deposits.
